(19) United States
(12) Patent Application Publication
(10) Pub. No.: US 2019/0298156 A1
(43) Pub. Date: Oct. 3, 2019

(54) ENDOSCOPE

(71) Applicant: OLYMPUS CORPORATION, Tokyo (JP)

(72) Inventor: Koji YAMAYA, Hachioji-shi (JP)

(73) Assignee: OLYMPUS CORPORATION, Tokyo (JP)

(21) Appl. No.: 16/448,116

(22) Filed: Jun. 21, 2019

Related U.S. Application Data
(63) Continuation of application No. PCT/JP2017/037860, filed on Oct. 19, 2017.

(30) Foreign Application Priority Data
Dec. 22, 2016 (JP) 2016-249768

(51) Int. Cl. A61B 1/00; G02B 23/24

(52) U.S. Cl. CPC A61B 1/00091; A61B 1/00098; A61B 1/018; G02B 23/2476; A61B 1/00147

(57) ABSTRACT

An endoscope includes a base, an operating portion, an elongated member, and a tube. The elongated member is inserted into the tube. The tube is elastically deformable between the one end and the other end of the tube. A side of closer to the one end of the tube is more compressively deformable along a longitudinal axis of the tube than a side closer to the other end of the tube.

### CROSS-REFERENCE TO RELATED APPLICATIONS

This application is a Continuation Application of PCT Application No. PCT/JP2017/037860, filed Oct. 19, 2017 and based upon and claiming the benefit of priority from prior Japanese Patent Application No. 2016-249768, filed Dec. 22, 2016, the entire contents of all of which are incorporated herein by reference.

### BACKGROUND OF THE INVENTION

### 1. Field of the Invention

The present invention relates to an endoscope.

### 2. Description of the Related Art

For example, Jpn. Pat. Appln. KOKAI Publication No. 6-315457 discloses an endoscope. The endoscope includes, on the distal end side of a channel through which an insertion tool such as a treatment tool is inserted, a mechanism that is configured to change the direction of the distal end of the treatment tool from a direction along the longitudinal axis of the insertion portion as needed. When a pulling member (elongated member) is moved in the axial direction by operating the operation portion of the endoscope, the operating portion coupled to the distal end of the pulling member disposed on the distal end portion of the insertion portion operates as appropriate around the axis of the support shaft as the pulling member moves.

### BRIEF SUMMARY OF THE INVENTION

According to one aspect of the present disclosure, there is provide an endoscope includes a base, an operating portion, an elongated member, and a tube. The base is provided on a distal end portion of an insertion portion. The base includes a passage configured to communicate a distal end side with a proximal end side. The operating portion is provided on a distal end side of the passage. The operating portion and is configured to be actuated with respect to the base. The elongated member includes a distal end side connected to the operating portion, and a proximal end side extending to a proximal end side of the base through the passage. The elongated member is configured to actuate the operating portion by moving in an axial direction of the elongated member. The elongated member is inserted into the tube. The tube including one end watertightly connected to the operating portion or the elongated member, and the other end watertightly connected to the base. The tube is configured to prevent a liquid from infiltrating the proximal end side of the passage from the distal end side of the passage through the passage. A side closer to the one end of the tube has a property of being more compressively deformable along a longitudinal axis of the tube than a side closer to the other end of the tube.

### BRIEF DESCRIPTION OF THE DRAWINGS

FIG. 1 is a schematic view showing an endoscope according to first to third embodiments;

FIG. 2 is a schematic perspective view showing the distal frame portion of the insertion portion of the endoscope and its neighboring portions according to the first to third embodiments;

FIG. 3A is a schematic partial sectional view showing a state in which the swing base of the distal frame portion of the insertion portion of the endoscope according to the first embodiment is arranged at a lowered position;

FIG. 3B is a schematic sectional view taken along line 3B-3B in FIG. 3A;

FIG. 4A is a schematic sectional view showing a state in which the swing base of the distal frame portion of the insertion portion of the endoscope according to the first embodiment is arranged at a raised position;

FIG. 4B is a schematic sectional view taken along line 4B-4B in FIG. 4A;

FIG. 5 is a schematic view showing a partial section different from that shown in FIG. 3A, in the state in which the swing base of the distal frame portion of the insertion portion of the endoscope according to the first embodiment is arranged at the lowered position;

FIG. 6A is a schematic view showing an operating portion, a tube, and a pulling member (elongated member) through while the tube extends with the distal end of the pulling member being fixed to the coupling portion of the operating portion, which are disposed in the distal frame portion of the insertion portion of the endoscope according to the first to third embodiments;

FIG. 6B is a schematic view showing a partial section of the swing base of the operating portion and sections of the coupling portion of the operating portion, the tube, and the pulling member, each of which is shown in FIG. 6A;

FIG. 7 is a schematic partial sectional view showing a state in which the pulling member extends through the tube and the distal end portion of the tube is to be fixed to the coupling portion while the coupling portion is coupled to the swing base of the operating portion;

FIG. 8 is a schematic perspective view showing a state in which a cover can be attached to and detached from the distal frame portion of the endoscope according to the first to third embodiments;

FIG. 9A is a schematic perspective view showing a state in which the inner and outer circumferential surfaces of the cover which are located near a deformed part of the tube are made to protrude outward while the cover is attached to the distal frame portion of the endoscope according to the first to third embodiments;

FIG. 9B is a schematic view showing the same partial section as that shown in FIG. 5, showing the distal frame portion to which the cover shown in FIG. 9A is attached;

FIG. 10 is a first modification of the schematic view of FIG. 6A showing a partial section of the swing base of the operating portion and sections of the coupling portion of the operating portion, the tube, and the pulling member;

FIG. 11A is a second modification of the schematic view of FIG. 6A showing a partial section of the swing base of the operating portion and sections of the coupling portion of the operating portion, the tube, and the pulling member;

FIG. 11B is a schematic partial sectional view showing a state in which the swing base of the distal frame portion of the insertion portion of the endoscope using the tube shown in FIG. 11A is arranged at the lowered position;

FIG. 12 is a third modification of the schematic view of FIG. 6A showing a partial section of the swing base of the operating portion and sections of the coupling portion of the operating portion, the tube, and the pulling member;

FIG. 13 is a fourth modification of the schematic view of FIG. 6A showing a partial section of the swing base of the operating portion and sections of the coupling portion of the operating portion, the tube, and the pulling member;

FIG. 14 is a fifth modification of the schematic view of FIG. 6A showing a partial section of the swing base of the operating portion and sections of the coupling portion of the operating portion, the tube, and the pulling member;

FIG. 15 is a sixth modification of the schematic view of FIG. 6A showing a partial section of the swing base of the operating portion and sections of the coupling portion of the operating portion, the tube, and the pulling member;

FIG. 16 is a partial sectional view showing a state in which the swing base of the distal frame portion of the insertion portion of the endoscope according to the second embodiment is arranged at a lowered position; and

FIG. 17 is a schematic view showing a partial section of the swing base of an operating portion and sections of the coupling portion of the operating portion, a tube, and a pulling member, which are disposed on the base member of the distal frame portion of the insertion portion of the endoscope according to the third embodiment.

### DETAILED DESCRIPTION

Modes for carrying out the present invention will be described below with reference to the accompanying drawings.

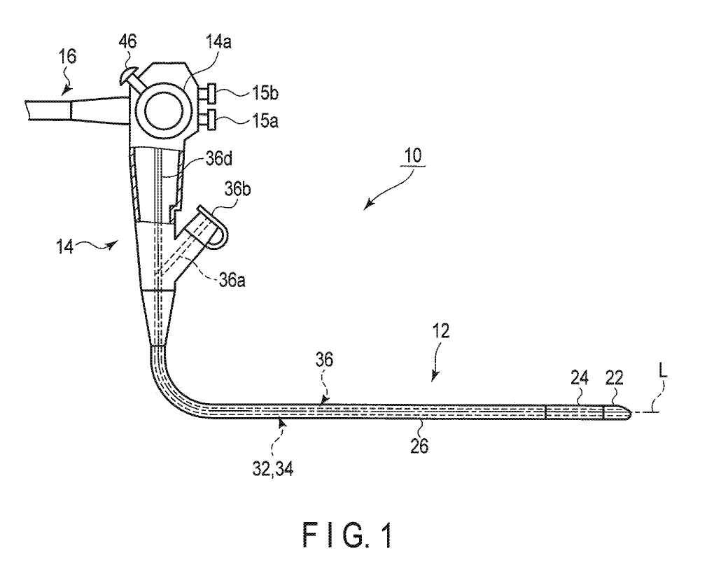

The first embodiment will be described with reference to FIGS. 1 to 7.

As shown in FIG. 1, an endoscope (insertion device) 10 according to the embodiment includes an insertion portion 12 inserted into a duct such as a lumen, an operation portion 14 provided on the proximal end of the insertion portion 12 and gripped by the user, and a universal cord 16 extending from the operation portion 14.

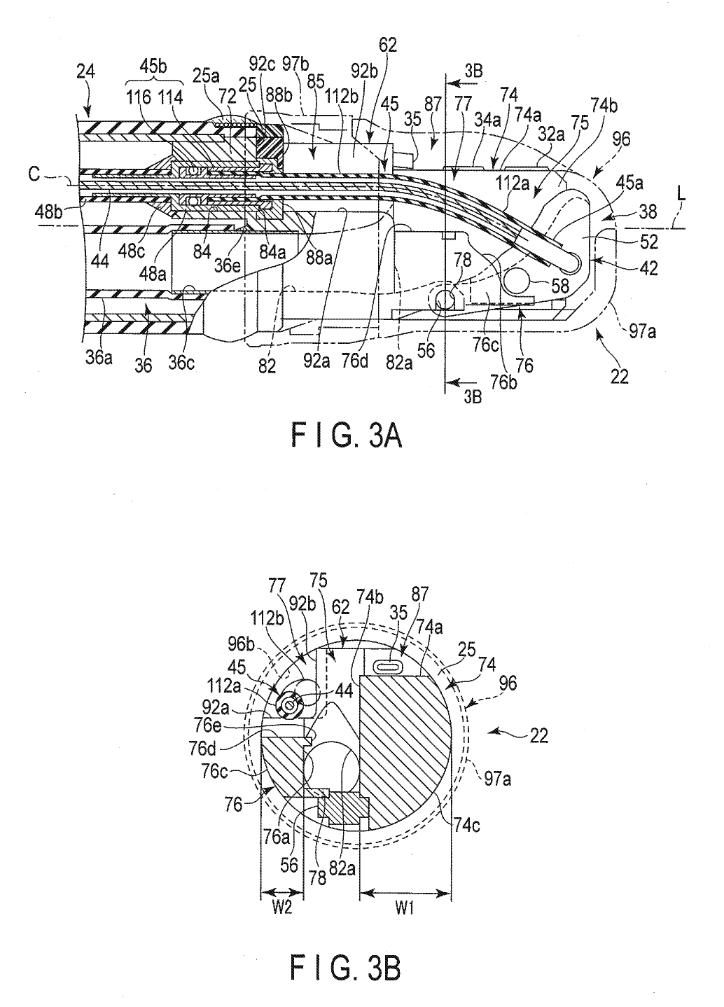

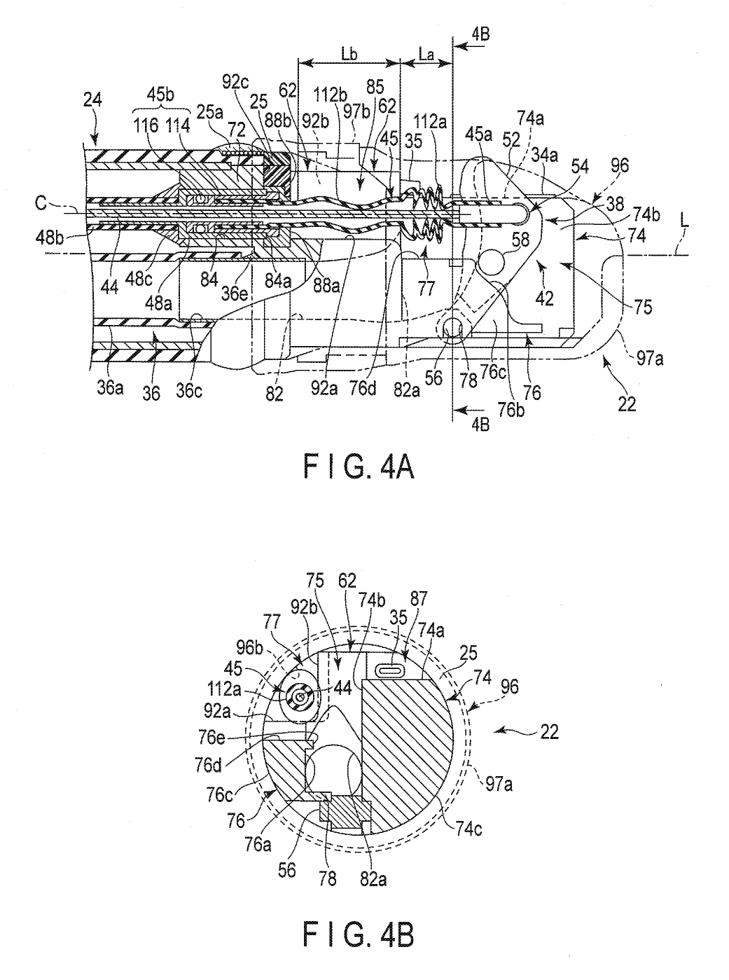

The insertion portion 12 has the distal end and the proximal end of the insertion portion 12. A longitudinal axis L is defined by the distal end and the proximal end of the insertion portion 12. The insertion portion 12 includes a distal frame portion 22, a bending portion 24, and a tube portion 26, sequentially from the distal end to the proximal end of the insertion portion 12. The tube portion 26 may be a so-called flexible scope having flexibility or a so-called rigid scope that maintains its straight state and has resistance to bending. A known mechanism allows the bending portion 24 to be bent in a plurality of directions such as two or four directions by operating a knob 14a of the operation portion 14. A swing base (raising base) 52 to be described later can be moved between a lowered position (initial position) shown in FIG. 3A and a swing position (maximum swing position) as the maximum raised position shown in FIG. 4A by operating a lever (swinging state adjustment portion) 46.

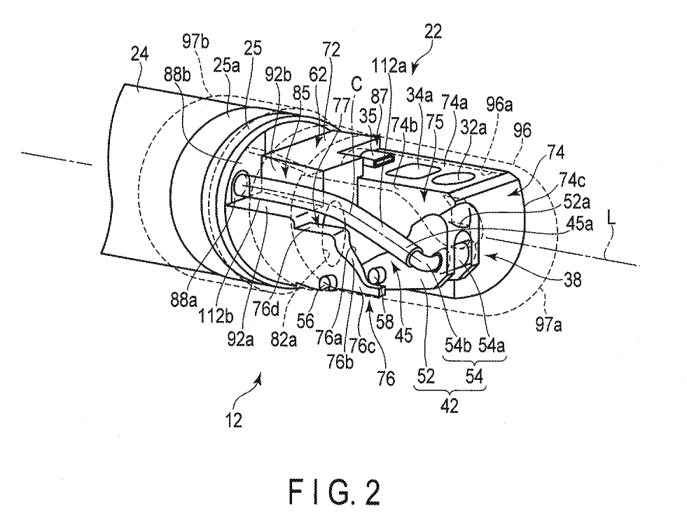

As shown in FIG. 2, an annular electrical insulating member 25 is fixed to the distal end of the bending portion 24. The electrical insulating member 25 is disposed on the outer circumference of a block-shaped base member 62 to be described later. A thread wound portion 25a is formed at a position adjacent to the proximal end side of the electrical insulating member 25.

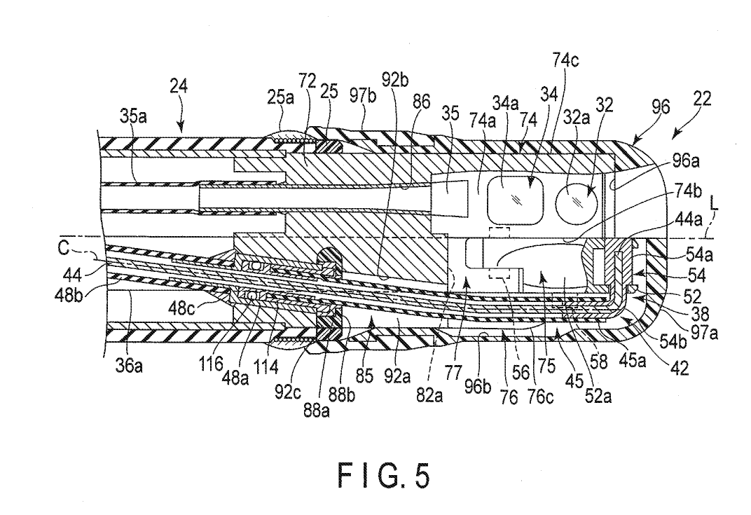

The endoscope 10 includes an illumination optical system 32, an observation optical system 34, and a treatment tool insertion channel 36. In addition, the endoscope 10 includes an air-supply/water-supply mechanism and a suction mechanism (not shown). The air-supply/water-supply mechanism includes a nozzle 35 and a tube 35a (see FIG. 5) on its distal end, and is operated by a button 15a of the operation portion 14 shown in FIG. 1. The suction mechanism communicates with the channel 36 and is operated by a button 15b of the operation portion 14 shown in FIG. 1.

The illumination optical system 32 and the observation optical system 34 are disposed inward from the distal frame portion 22, the bending portion 24, and the tube portion 26 of the insertion portion 12, the operation portion 14, and universal cord 16 of the endoscope 10. The illumination optical system 32 includes an illumination window 32a on the distal frame portion 22. The observation optical system 34 includes an observation window 34a on the distal frame portion 22. The following is a case in which the observation optical system 34 is formed as a side-viewing endoscope for observation in a direction perpendicular to a longitudinal axis L. However, the observation optical system 34 may be formed as a direct-viewing endoscope for observation in a direction along the longitudinal axis L. Alternatively, the observation optical system 34 may be formed as an oblique-viewing endoscope for observation in a direction away from the longitudinal axis L. The side-viewing, direct-viewing, and oblique-viewing observation optical systems 34 each are known. In this case, the endoscope 10 including the side-viewing observation optical system 34 will be described.

A distal end of the channel 36 is opened in the distal frame portion 22 of the insertion portion 12 of the endoscope 10. A proximal end of the channel 36 is opened near the proximal end portion of the tube portion 26 of the insertion portion 12 or in the operation portion 14. In this case, as shown in FIG. 1, the proximal end of the channel 36 has an opening (not shown) in the operation portion 14, and a forceps plug 36b is detachable to the opening through a mouth ring (not shown). The distal end of a tube 36a of the channel 36 is fixed to the distal frame portion 22 through a mouth ring 36c. Note that the tube 36a of the channel 36 branches to a known suction path 36d inside, for example, the operation portion 14. The suction path 36d is coupled to the button 15b. Pressing the button 15b will discharge sucked substances from a distal end opening 82a (to be described later) of the distal end of the channel 36 through the mouth ring 36c, the tube 36a, the suction path 36d, and the universal cord 16.

As described above, in this embodiment, the distal frame portion 22 is formed as a side-viewing type with a different observation direction relative to a direction along the longitudinal axis L of the insertion portion 12. The endoscope 10 includes a swinging mechanism 38 that can properly adjust the direction of a treatment tool (not shown) or the like inserted into the channel 36 at the distal frame portion 22 so as to set the tool in the visual field of the observation optical system 34.

A distal end of the swinging mechanism 38 is located in the distal frame portion 22 of the insertion portion 12 of the endoscope 10. A proximal end of the swinging mechanism 38 is located in the operation portion 14. The swinging mechanism 38 includes an operating portion 42 supported by the base member 62 (to be described later), an elongated member (to be referred to as a pulling member hereinafter) 44 (see FIG. 3A or the like) that moves in the axial direction to actuate the operating portion 42, and the lever 46 (see FIG. 1) supported by the operation portion 14. The operating portion 42 is provided on the distal end portion of the insertion portion 12 and on the distal end side of a second cylindrical surface 84 and a mouth ring 48a (which will be described later), and is operated relative to the base member 62. As the pulling member 44, for example, a wire or rod-like member (rod) having flexibility is used. The pulling member 44 extends to the proximal end side of the base member 62 through the passage (mouth ring 48a) (see FIG. 3A or the like) of the base member 62 (to be described later) at the distal end portion of the insertion portion 12. The pulling member 44 then extends to the operation portion 14 through the insides of the bending portion 24 and the tube portion 26. The length of the pulling member 44 is adjusted. A distal end portion 44a on the distal end side of the pulling member 44 is connected to the operating portion 42. The proximal end portion (not shown) on the proximal end side of the pulling member 44 is supported by the lever 46 of the operation portion 14. Although described in detail later, the operating portion 42 is part of the distal frame portion 22. The distal end portion 44a of the pulling member 44 and its neighboring portions are also part of the distal frame portion 22.

The operating portion 42 includes the swing base (raising base) 52 including a guide path 52a for a treatment tool and a coupling portion 54 coupled to the swing base 52. The swing base 52 is formed into an almost triangular prism shape. The guide path 52a of the swing base 52 guides a treatment tool (not shown) protruding from the distal end opening 82a of a first cylindrical surface 82 forming a first through hole (channel) (to be described later), and changes the direction of the distal end of the treatment tool to a direction deviating from a direction along the longitudinal axis L of the insertion portion 12. As shown in FIGS. 3A to 7, the swing base 52 is provided with, for example, a pivot shaft 56 that is perpendicular to the longitudinal axis L and also perpendicular to the observation direction and a guide pin 58 that is supported on a guide surface 76b (to be described later) and guides the swing base 52 so as to allow it to move in a predetermined range. The swing base 52 is preferably integrally provided with the pivot shaft 56 and the guide pin 58.

Note that the pivot shaft 56 of the swing base 52 of the operating portion 42 need not always be perpendicular to the longitudinal axis L and is allowed to shift as needed. In addition, the axial direction of the pivot shaft 56 of the swing base 52 of the operating portion 42 need not always be perpendicular to the observation direction of the observation optical system 34 and is allowed to shift as needed.

As shown in FIGS. 2 to 3B, the distal frame portion 22 provided at the distal end portion of the insertion portion 12 includes the block-shaped base member 62. The base member 62 is formed by, for example, cutting a cylindrical column made of a hard material such as a metal like stainless steel or a hard resin. The base member 62 generally includes an almost columnar base 72 and first and second convex portions 74 and 76 extending from the base 72 to the distal end side along the longitudinal axis L. The outer circumference of the base 72 of the base member 62 is covered by the distal end portion of a rubber tube on the outermost circumference of the bending portion 24. The thread wound portion 25a has the distal end portion of the rubber tube of the bending portion 24 disposed in tight contact with the outer circumference of the base 72 of the base member 62. The electrical insulating member 25 is disposed at the distal end of the rubber tube of the bending portion 24.

The first convex portion 74 is provided with the illumination window 32a of the illumination optical system 32 and the observation window 34a of the observation optical system 34. The illumination window 32a of the illumination optical system 32 and the observation window 34a of the observation optical system 34 are directed in a direction perpendicular to the longitudinal axis L. The base 72 is provided with the nozzle 35 on the proximal end side of the illumination window 32a of the illumination optical system 32 and the observation window 34a of the observation optical system 34. The opening of the nozzle 35 is directed to the illumination window 32a of the illumination optical system 32 and the observation window 34a of the observation optical system 34. The nozzle 35 can discharge a liquid such as physiological saline toward the observation window 34a and the illumination window 32a and blow off deposits on the observation window 34a and the illumination window 32a by supplying air.

The first convex portion 74 includes an arrangement surface 74a on which illumination window 32a of the illumination optical system 32 and the observation window 34a of the observation optical system 34 are arranged, a defining surface (flat surface) 74b that defines the moving direction of the swing base 52, and an outer circumferential surface 74c. The arrangement surface 74a is preferably formed as a surface that extends along the longitudinal axis L and is parallel to the pivot shaft 56 of the operating portion 42. The defining surface 74b is preferably a flat surface that extends along the longitudinal axis L and is perpendicular to the pivot shaft 56 of the operating portion 42. In this case, the defining surface 74b is perpendicular to the arrangement surface 74a. The outer circumferential surface 74c is formed as a curved surface of a cylindrical column.

The second convex portion 76 is separated from the defining surface 74b of the first convex portion 74. The second convex portion 76 includes a defining surface 76a that faces the defining surface 74b of the first convex portion 74, a guide surface 76b that guides the pivotal movement of the swing base 52, an outer circumferential surface 76c, and an extension surface 76d extending from the base 72. The defining surface 76a of the second convex portion 76 is preferably a flat surface parallel to the defining surface 74b of the first convex portion 74. The outer circumferential surface 76c is formed as a curved surface of a cylindrical column. A projection 76e is formed on the defining surface 76a of the second convex portion 76. The projection 76e comes into contact with the swing base 52 when the swing base 52 is at the swing position (raised position). That is, the projection 76e defines the maximum swing position (maximum raised position) at which the swing base 52 swings (rises) most.

A space 75 is formed between the defining surface 74b of the first convex portion 74 and the defining surface 76a of the second convex portion 76. The operating portion 42 is arranged in the space 75. The space 75 allows the operating portion 42 to operate in a predetermined range. In the space 75, in particular, the swing base 52 of the operating portion 42 is arranged and operated in a predetermined range. A support portion 78 that supports the pivot shaft 56 of the swing base 52 is disposed on the defining surface 74b of the first convex portion 74 and the defining surface 76a of the second convex portion 76 at positions separated from the arrangement surface 74a of the first convex portion 74 and the extension surface 76d of the second convex portion 76. That is, the base member 62 includes the support portion 78 that supports the operating portion 42 so as to allow it to operate. In this case, the support portion 78 is formed into an almost U shape. As shown in FIG. 2, the swing base 52 is disposed in the space 75, and the pivot shaft 56 is disposed on the support portion 78. A cover 96 (to be described later) is attached to the outsides of the base member 62 and the operating portion 42. The cover 96 prevents the pivot shaft 56 of the swing base 52 from slipping off from the support portion 78 of the base member 62.

Note that the proximal end portion of the operating portion 42 protrudes more to the distal end side along the longitudinal axis L with respect to the distal end of the base member 62 while the operating portion 42 is arranged at the lowered position shown in FIG. 3A. This makes it easier to clean a fitting portion 52b (to be described later) of the swing base 52.

The guide pin 58 of the swing base 52 is placed on the guide surface 76b of the second convex portion 76 while the swing base 52 is disposed in the space 75 and the pivot shaft 56 is disposed on the support portion 78. The guide surface 76b is formed as a proper curved surface, and moves the guide pin 58 between the position shown in FIGS. 2 and 3A and the position shown in FIG. 4A while supporting the guide pin 58 of the swing base 52. That is, the operating portion 42 operates around the support portion 78 in accordance with a pulling force from the pulling member 44.

A maximum width (height) W2 of the defining surface 76a of the second convex portion 76 in a direction perpendicular to the longitudinal axis L is, for example, about half of a maximum width (height) W1 of the defining surface 74b of the first convex portion 74 in a direction perpendicular to the longitudinal axis L. A movement space 77 which is continuous with the space 75 described above and in which the pulling member 44 and the tube (tubular elastic member) 45 covering the pulling member 44 move is formed in a region which is adjacent to a side where the extension surface 76d is directed to the second convex portion 76.

The base 72 of the base member 62 includes the first cylindrical surface 82 in which a first through hole (channel hole) through which a treatment tool extends is formed, the second cylindrical surface 84 in which a second through hole (a passage for the pulling member 44 of the swinging mechanism 38) inclined with respect to, for example, the longitudinal axis L is formed, and a third cylindrical surface 86 in which a third through hole (a passage for the nozzle 35) along, for example, the longitudinal axis L is formed. The first cylindrical surface 82, the second cylindrical surface 84, and the third cylindrical surface 86 each are allowed to have an appropriate shape, and preferably have, for example, cylindrical shape. The inner diameter of the first cylindrical surface 82 is preferably larger than that of the second cylindrical surface 84. The inner diameter of the first cylindrical surface 82 is preferably larger than that of the third cylindrical surface 86.

The first cylindrical surface 82 extends through the base member 62 so as to be parallel or almost parallel to, for example, the longitudinal axis L. More specifically, the first cylindrical surface 82 extends through the base 72 of the base member 62. That is, the first cylindrical surface 82 extends along, for example, the longitudinal axis L so as to make the distal end side of the base 72 of the base member 62 communicate with the proximal end side. The first and second convex portions 74 and 76 define the appropriate space 75 together with the cover 96 on the distal end side of the first cylindrical surface 82.

The second cylindrical surface (introduction hole) 84 extends through the base member 62 so as to be inclined relative to, for example, the longitudinal axis L. More specifically, the second cylindrical surface 84 extends through the base 72 of the base member 62. That is, the second cylindrical surface 84 makes the distal end side of the base 72 of the base member 62 communicate with the proximal end side. The base 72 and the cover 96 cooperatively define an appropriate space 85 on the distal end side of the second cylindrical surface 84. The space 85 is continuous with the proximal end side of the space 75 and the space 77 described above along the longitudinal axis L. Because the through hole direction of the second cylindrical surface 84 is inclined relative to the longitudinal axis L, the length (the length of an elastic member 112 (to be described later)) between a distal end (one end) 45a of the tube 45 and a proximal end (the other end) 45b can be maximized in the distal frame portion 22, and the deformation amount of the elastic member 112 per unit volume can be reduced. Depending on the selection of a material for the elastic member 112, the through hole direction of the second cylindrical surface 84 preferably extends along the longitudinal axis L.

Note that the through hole direction of the second cylindrical surface 84 coincides or almost coincides with the axial direction of the central axis (longitudinal axis) C of the pulling member 44.

The third cylindrical surface 86 extends though the base member 62 along, for example, the longitudinal axis L. More specifically, the third cylindrical surface 86 extends through the base 72 of the base member 62. That is, the third cylindrical surface 86 extends along, for example, the longitudinal axis L so as to make the distal end side of the base 72 of the base member 62 communicate with the proximal end side. The first convex portion 74 and the cover 96 cooperatively define an appropriate space 87, on the distal end side of the third cylindrical surface 86, in which the distal end of the nozzle 35 is disposed.

The first cylindrical surface 82 forms the distal end opening 82a of the channel 36. The mouth ring 36c is fixed to the first cylindrical surface 82. The channel tube 36a is fixed to the proximal end portion of the mouth ring 36c. An adhesive agent 36e is, for example, annularly applied between the proximal end of the base 72 of the base member 62 and the mouth ring 36c. This prevents a fluid (including a liquid and a gas) from leaking from the mouth ring 36c to the proximal end side of the mouth ring 36c through the outer circumferential surface of the mouth ring 36c and the base 72. Note that in this embodiment, the distal end 82a of the first cylindrical surface 82 is disposed closer to the distal end side along the longitudinal axis L than the distal end of the mouth ring 36c.

As described above, the second cylindrical surface 84 is formed so as to be inclined relative to the longitudinal axis L. The pulling member 44 of the swinging mechanism 38 extends through the second cylindrical surface 84. The mouth ring (passage) 48a is fixed to the second cylindrical surface 84. The mouth ring 48a forms a through hole (introduction hole) and forms a passage through which the pulling member 44 extends. That is, the base member 62 includes the mouth ring 48a as a passage that makes the distal end side communicate with the proximal end side. A tube 48b is fixed to the proximal end portion of the mouth ring 48a. For example, an adhesive agent 48c is annularly applied to the proximal end of the second cylindrical surface 84 of the base 72 of the base member 62 and between the mouth ring 48a and the tube 48b. This prevents a fluid (including a liquid and a gas) from leaking from the distal end of the mouth ring 48a to the proximal end side of the mouth ring 48a through between the outer circumferential surface of the mouth ring 48a and the base 72.

While the proximal end portion of the tube 45 is disposed on the second cylindrical surface 84, an adhesive agent 88a and a retaining plate (protective plate) 88b are disposed on the mouth ring 48a. The adhesive agent 88a and the retaining plate 88b are preferably disposed inward from the electrical insulating member 25. The adhesive agent 88a and the retaining plate 88b are disposed on a distal end 84a of the second cylindrical surface 84 of the base 72. With this arrangement, the adhesive agent 88a and the retaining plate 88b prevent the proximal end portion of the tube 45 from slipping off from the mouth ring 48a. In addition, the adhesive agent 88a prevents a liquid from infiltrating from the distal end side into the proximal end side through between the outer circumferential surface of a mouth ring 114 (to be described later) and the inner circumferential surface of the mouth ring 48a and between the outer circumferential surface of the mouth ring 48a and the second cylindrical surface 84, that is, between the outer circumferential surface of the mouth ring 114 and the second cylindrical surface 84.

The base 72 includes a first wall surface 92a, a second wall surface 92b, and a third wall surface 92c, on the distal end side of the second cylindrical surface 84. The first wall surface (bottom surface) 92a, the second wall surface (side surface) 92b, and the third wall surface (proximal end surface) 92c are formed at positions closer to the proximal end side than the distal end opening 82a of the first cylindrical surface (channel hole) 82 along the longitudinal axis L. The first wall surface 92a, the second wall surface 92b, and the third wall surface 92c form the space (gap) 85 between an opening edge 96a of the cover 96 (to be described later) and/or an inner circumferential surface 96b. In this embodiment, as shown in FIGS. 3A and 4A, the first wall surface 92a is formed parallel to the arrangement surface 74a of the first convex portion 74 and the extension surface 76d of the second convex portion 76. The first wall surface 92a is located between the arrangement surface 74a of the first convex portion 74 and the extension surface 76d of the second convex portion 76 in a direction perpendicular to the longitudinal axis L in FIGS. 3A and 4A.

Note that the nozzle 35 extends through the third cylindrical surface 86 and is fixed to the third cylindrical surface 86. The tube 35a is fixed to the proximal end of the nozzle 35.

The distal end (distal end opening) 82a of the first cylindrical surface 82 is formed closer to the distal end side along the longitudinal axis L with respect to the distal end 84a of the second cylindrical surface 84. The distal end opening 82a of the first cylindrical surface 82 is formed on the proximal end side of the swing base 52. Note that because the distal end opening 82a of the first cylindrical surface 82 is formed along the longitudinal axis L so as to be closer to the distal end side with respect to the distal end 84a of the second cylindrical surface 84, in the distal frame portion 22, the length of the elastic member 112 (to be described later) of the tube 45 can be maximized, and the deformation amount of the elastic member 112 per unit volume can be reduced when the elastic member 112 is compressed along a central axis C.

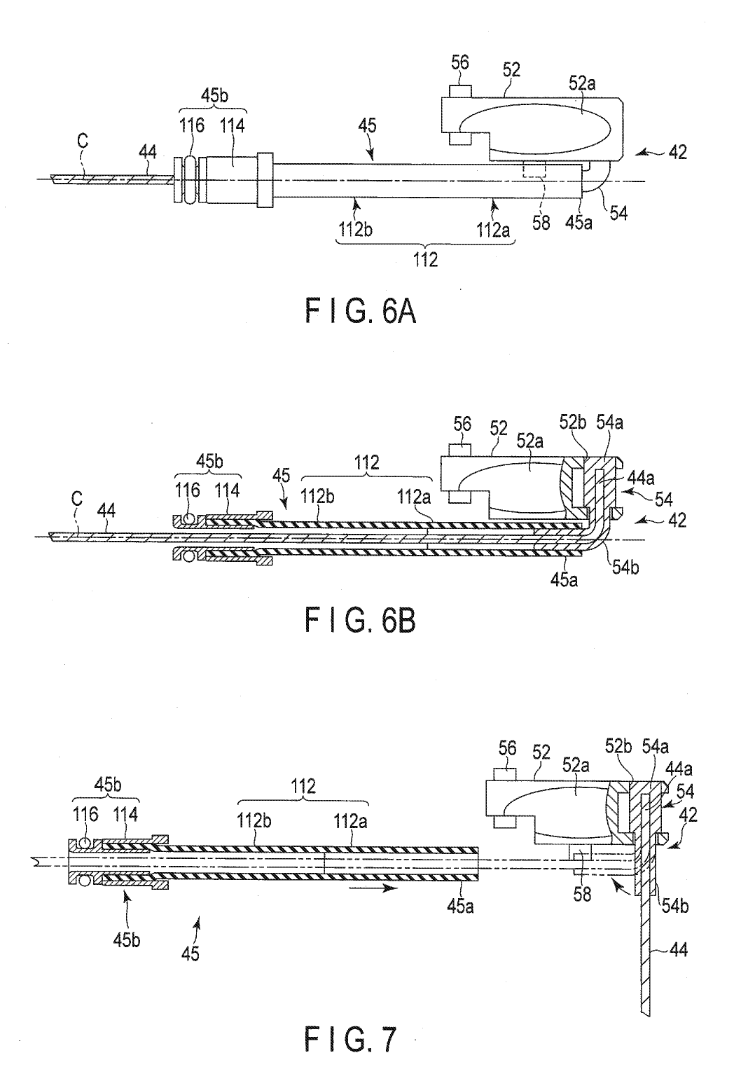

As shown in FIGS. 6A to 7, the swing base 52 of the operating portion 42 includes the fitting portion 52b in which the coupling portion 54 is fitted. The fitting portion 52b of the swing base 52 is formed into a concave portion or through hole in which the coupling portion 54 is disposed. In this case, the fitting portion 52b extends through the swing base 52 in a direction perpendicular to the longitudinal axis L.

As shown in FIG. 7, the coupling portion 54 can rotate relative to the swing base 52 as appropriate as shown in FIGS. 3A and 4A, while the distal end portion 44a of the pulling member 44 is fixed. As shown in FIGS. 3A, 4A, 6A to 7, the coupling portion 54 includes, for example, a bottomed cylindrical body 54a having one end closed and a tubular body 54b having an inner circumferential surface continuous with the inner circumferential surface of the bottomed cylindrical body 54a and an outer diameter smaller than that of the bottomed cylindrical body 54a.

The distal end portion 44a of the pulling member 44 is fixed to the bottomed cylindrical body 54a of the coupling portion 54 by, for example, swaging. As shown in FIGS. 6B and 7, the fitting portion 52b of the swing base 52 is formed such that the opening diameter on the upper side of the drawing surface of each of FIGS. 6B and 7 is larger than the opening diameter on the lower side. The tubular body 54b of the coupling portion 54 is fitted in the fitting portion 52b from the upper side to the lower side in FIGS. 6B and 7. In this state, the tubular body 54b is bent in a direction along the longitudinal axis L. This allows the coupling portion 54 to rotate about a rotation axis (a rotation axis perpendicular to the longitudinal axis L) relative to the swing base 52 as appropriate, but prevents the coupling portion 54 from slipping off from the swing base 52. Accordingly, while the distal end portion 44a of the pulling member 44 is fixed, the coupling portion 54 is bent in a direction along the central axis C of the pulling member 44 on the outside of the swing base 52.

The outer circumference of the pulling member 44 is covered with the cylindrical tube 45. That is, the pulling member 44 is inside the tube 45. The central axis of the cylindrical tube 45 coincides or almost coincides with the central axis C of the pulling member 44. The tube 45 includes the expandable cylindrical elastic member 112 that is elastically deformed along the axial direction of the pulling member 44, the mouth ring 114 fixed to the proximal end (the other end) of the elastic member 112, and an O-ring 116 disposed on the outer circumference of the mouth ring 114.

The distal end (one end) 45a of the elastic member 112 of the tube 45 is fixed to the tubular body 54b of the coupling portion 54 with, for example, adhesive bonding in this embodiment. The inner circumferential surface of the distal end 45a of the elastic member 112 is entirely in tight contact with the outer circumferential surface of the tubular body 54b of the coupling portion 54. This prevents a liquid or gas from infiltrating the inside of the elastic member 112 from the distal end (one end) 45a of the elastic member 112 of the tube 45. That is, one end 45a of the tube 45 is watertightly connected to the operating portion 42.

The other end 45b of the tube 45 is formed by the elastic member 112, the mouth ring 114, and the O-ring 116. The mouth ring 114 fixes the proximal end of the elastic member 112 by, for example, clamping. The mouth ring 114 may be integrally formed or formed from a plurality of members, for example, two members. The O-ring 116 prevents a liquid from moving from between the outer circumferential surface of the mouth ring 114 and the inner circumferential surface of the O-ring 116 along the axial direction of the mouth ring 114. That is, even if the adhesive agent 88a cracks to let a liquid infiltrate from the distal end side into between the outer circumferential surface of the mouth ring 114 and the second cylindrical surface 84, the O-ring 116 can reliably prevent the infiltration of the liquid. The proximal end (the other end) 45b of the tube 45 is fitted to the inner circumferential surface (annular circumference) of the mouth ring 48a fixed to the base member 62 of the distal frame portion 22. It is preferable that the inner diameter of the mouth ring 48a is slightly larger than the outer diameter of the mouth ring 114 of the other end 45b of the tube 45 and is slightly smaller than the outer diameter of the O-ring 116. Bringing the outer circumferential surface of the O-ring 116 into tight contact with the inner circumferential surface of the mouth ring 48a can prevent a liquid or gas from flowing to the proximal end side even if the adhesive agent 88a cracks or collapses to damage the watertight structure at a portion where cracking or collapsing has occurred. That is, the other end 45b of the tube 45 is watertightly connected to the base member 62 through the mouth ring 48a. This makes the other end 45b of the tube 45 have a structure that doubly prevents a liquid from infiltrating the proximal end side from the distal end side through the passage (through hole) 48a with the adhesive agent 88a and the O-ring 116.

As described above, while the proximal end (the other end) 45b of the tube 45 is fitted to the inner circumferential surface of the mouth ring 48a, the adhesive agent 88a and the retaining plate 88b are disposed on the base 72. This maintains the fitted state between the other end 45b of the tube 45 and the mouth ring 48a even if a force acts to release the fitting between the other end 45b of the tube 45 and the mouth ring 48a during the use of the endoscope 10. Note that the length of a portion, of the elastic member 112, which protrudes from the retaining plate 88b to the distal end side (the effective length of the deformable portion of the elastic member 112 except for one end 45a and the other end 45b) is preferably, for example, about 20 mm.

The elastic member 112 of the tube 45 is formed from a resin material that can be deformed as the pulling member 44 is pulled. The entire portion of the elastic member 112 between one end 45a and the other end 45b, in particular, is preferably deformable. The elastic member 112 includes a distal end side region 112a and a proximal end side region 112b. In this embodiment, the proximal end of the distal end side region 112a is continuous with the distal end of the proximal end side region 112b. As described above, when the portion of the elastic member 112 which protrudes from the retaining plate 88b to the distal end side has a length of about 20 mm, the distal end side region 112a and the proximal end side region 112b each preferably have a length of, for example, about 10 mm. In addition, in the embodiment, the elastic member 112 has a constant thickness from the distal end to the proximal end. On the other hand, the distal end side region 112a and the proximal end side region 112b of the elastic member 112 may be formed from different materials and each may have deformability adjusted by composition adjustment. The distal end side region 112a of the elastic member 112, which is close to one end 45a, has a property of being more deformable than the proximal end side region 112b, which is close to the other end 45b. For example, the undeformability of the proximal end side region 112b is preferably higher by 10% to 30%, preferably 20%, than that of the distal end side region 112a. For example, the distal end side region 112a of the elastic member 112 is preferably formed from a fluorine resin or silicone resin, whereas the proximal end side region 112b is preferably formed from an urethane resin. In addition, for example, the proximal end side region 112b of the elastic member 112 and the boundary between the proximal end side region 112b and the distal end side region 112a may be formed by two-color molding (different material molding) using the above resin materials as needed. Resin materials are properly selected for the elastic member 112 so as to maintain the deformability of the distal end side region 112a higher than that of the proximal end side region 112b when a compression force is exerted along the central axis C.

In this manner, the elastic member 112 is formed such that the distal end side region 112a located close to one end 45a is more deformable upon compression along the central axis C of the tube 45 with respect to the proximal end side region 112b located close to the other end 45b. Accordingly, when the elastic member 112 is compressed along the central axis C, the reduction ratio of the length of the distal end side region 112a as compared with the length before compression is higher than that of the proximal end side region 112b. That is, the side of the elastic member 112 which is connected to the operating portion 42 is more deformable than the side of the elastic member 112 which is connected to the periphery of the through hole.

The distal end side region 112a of the elastic member 112 is preferably provided with a creasing tendency in advance so as to form a plurality of creases when being compressively deformed along the axial direction of the elastic member 112 as shown in FIG. 4A. For example, when the distal end side region 112a is compressively deformed along the axial direction of the elastic member 112, a plurality of creases (a plurality of ridges and valleys) are preferably formed. Assume that when the distal end side region 112a of the elastic member 112 is compressively deformed along the axial direction of the elastic member 112, only one ridge is formed. In this case, the distal end side region 112a can have a large maximum outer diameter. In contrast to this, forming a plurality of creases, that is, a plurality of ridges together with a plurality of valleys instead of only one ridge, can reduce the maximum outer diameter of the distal end side region 112a. Assume that in the following description, when the distal end side region 112a of the elastic member 112 is compressively deformed along the axial direction of the elastic member 112, a plurality of creases are formed.

As shown in FIG. 7, while the pulling member 44 extends through the tube 45, the coupling portion 54 is fixed to the distal end portion 44a of the pulling member 44, and the tubular body 54b is bent. One end 45a of the tube 45 is fixed to the tubular body 54b of the coupling portion 54. Subsequently, the other end 45b of the tube 45 is fitted in the mouth ring 48a, and the swing base 52 is supported on the base member 62. As shown in FIGS. 3A, 4A, and 5, the adhesive agent 88a and the retaining plate 88b are disposed on the mouth ring 48a to maintain the fitted state between the other end 45b of the tube 45 and the mouth ring 48a. That is, the outer circumferential surface of a portion, of the elastic member 112 of the tube 45, which is close to the other end 45b is fixed to the base member 62 with the adhesive agent 88a and the retaining plate 88b which are disposed between the outer circumferential surface and the mouth ring 48a disposed on the second cylindrical surface 84. The adhesive agent 88a is circumferentially applied between the distal end of the mouth ring 48a and the distal end 84a of the passage 84. This prevents a liquid and a gas from infiltrating the inside of the bending portion 24 and the tube portion 26 (see FIG. 1) of the insertion portion 12 through not only the inside (on the O-ring 116 side) of the mouth ring 48a but also the outside of the mouth ring 48a along the outer circumference of the tube 45. In this manner, the fixing structures of the distal end and the proximal end of the tube 45 prevent a liquid and a gas from infiltrating the inside of the bending portion 24 and the tube portion 26 (see FIG. 1) of the insertion portion 12 from the outer circumferential surface of the tube 45.

In this case, when the elastic member 112 of the tube 45 has a natural length, the distal end side region 112a can be disposed in the space 75 and the space 77, and the proximal end side region 112b can be disposed in the space 77 and the space 85. Accordingly, the distal end side region 112a of the elastic member 112 of the tube 45 is arranged between the distal end opening 82a of the first cylindrical surface 82 and the operating portion 42 along the longitudinal axis L. In addition, it is preferable that only the proximal end side region 112b of the elastic member 112 is arranged, without the distal end side region 112a, between the distal end 84a of the passage 84 and the distal end opening 82a of the channel hole 82.

When the elastic member 112 of the tube 45 has a natural length, the outer circumferential surface of the elastic member 112 preferably does not come into contact with any of the first wall surface 92a, the second wall surface 92b, and the distal end face of the retaining plate 88b on the distal end side of the third wall surface 92c.

In addition, the elastic member 112 preferably does not come into any of the first convex portion 74 and the second convex portion 76.

The outer diameter of the pulling member 44 is, for example, about 0.5 mm. The inner diameter of the elastic member 112 is, for example, about 0.8 mm, and the outer diameter of the elastic member 112 is, for example, about 1.3 mm to 1.5 mm. The clearance between the outer circumferential surface of the pulling member 44 and the inner circumferential surface of the elastic member 112 is, for example, about 0.2 mm.

The illumination window 32a of the illumination optical system 32, the observation window 34a of the observation optical system 34, the mouth ring 36c of the distal end portion of the channel 36, the operating portion 42 of the swinging mechanism 38, the pulling member 44, the tube 45, the mouth ring 48a, and the like are properly attached to the base member 62. In this state, the distal end cover 96 is attached to the outer circumferences of these components to form the distal frame portion 22.

The cover 96 is preferably formed from a resin material having an electrical insulation property and/or a rubber material having an electrical insulation property. Referring to FIGS. 2 to 5, the cover 96 is constituted by a main body 97a made of a resin material and a cover 97b made of a rubber material. Obviously, however, the cover 96 may be integrally formed.

The cover 96 covers the outer circumferential surface of the base 72 of the base member 62 of the distal frame portion 22, the outer circumferential surface 74c of the first convex portion 74, and the outer circumferential surface 76c of the second convex portion 76. The cover 96 includes the opening edge 96a and exposes the illumination window 32a of the illumination optical system 32 and the observation window 34a of the observation optical system 34 toward the outside of the endoscope 10. In this embodiment, the cover 96 also exposes the space 75, in which the swing base 52 is disposed, and the space 77 and the space 85, in which the tube 45 is disposed, toward the outside of the endoscope 10 on the upper side of the drawing surface of each of FIGS. 3A to 4B. Obviously, only part of each of the space 75, the space 77, and the space 85 is preferably exposed toward the outside of the endoscope 10.

The space 75, the space 77, and the space 85 define the movable ranges of the pulling member 44 and the elastic member 112, together with the inner circumferential surface 96b of the cover 96.

The operation of the endoscope 10 according to this embodiment will be described next.

When the lever 46 supported by the operation portion 14 shown in FIG. 1 is operated, the operating portion 42 supported by the base member 62 of the distal frame portion 22 moves in interlocking with the lever through the pulling member 44. When the lever 46 is raised to the highest position (first position), the operating portion 42 is arranged at the neutral position (lowered position) shown in FIGS. 3A and 3B. At this time, the pulling force on the pulling member 44 is released, and the pulling member 44 moves to the most distal end side. As the lever 46 is pressed down, the pulling member 44 is pulled toward the proximal end side, and the operating portion 42 pivots about the pivot shaft 56. At this time, when the lever 46 is pressed down to the lowest position (second position), the operating portion 42 is arranged at a swing position (raised position) shown in FIGS. 4A and 4B. The lever 46 shown in FIG. 1 is then arranged at the first position, and the swing base 52 is set at the lowered position shown in FIGS. 3A and 3B. At this time, the elastic member 112 of the tube 45 outside the pulling member 44 has a natural length without any crease. In this case, while the tube 45 is properly assembled with the distal frame portion 22, the distal end side region 112a of the elastic member 112 is preferably configured to form creases more easily than the proximal end side region 112b. Note that when the lever 46 is at the first position, the elastic force of the pulling member 44, the tube 45, and the like also act to prevent the lever 46 from unintentionally moving from the first position to the second position, thereby preventing the operating portion 42 from unintentionally swinging.

In this state, the insertion portion 12 is inserted into a given lumen with the distal frame portion 22 leading. While the distal end of the insertion portion 12 is arranged at a desired position in a desired direction, a treatment tool (not shown) is inserted from the proximal end of the channel 36 toward the distal end. The distal end of the treatment tool is placed in the guide path 52a of the swing base 52 through the distal end opening 82a of the first cylindrical surface 82. As the treatment tool (not shown) is inserted from the proximal end of the channel 36 toward the distal end, the distal end of the treatment tool exceeds the guide path 52a of the swing base 52 and the opening edge 96a on the distal end side of the distal end cover 96 to protrude from the distal frame portion 22.

When the lever 46 is moved from the first position to the second position to pull the pulling member 44, the coupling portion 54 is pulled by the pulling member 44.

This causes the swing base 52 coupled to the coupling portion 54 to pivot about the axis of the pivot shaft 56 and move to the swing position (raised position) shown in FIGS. 4A and 4B. At this time, the guide pin 58 of the swing base 52 moves along the guide surface 76b. The swing base 52 comes into contact with the projection 76e of the second convex portion 76. Accordingly, the swing base 52 is located at the swing position (raised position) shown in FIGS. 4A and 4B. At this time, the distal end of the treatment tool (not shown) is bent by the guide path 52a of the swing base 52 to be directed in the observation direction of the observation window 34a of the observation optical system 34.

One end 45a of the tube 45 through which the pulling member 44 extends is movable relative to the base member 62. In contrast to this, the other end 45b is fixed while being fitted in the base member 62, and hence cannot move.

Accordingly, when the pulling member 44 is pulled by operating the lever 46 to move the swing base 52 from the lowered position to the swing position, one end 45a of the tube 45 moves to the other end 45b. At this time, a compression force is applied to the elastic member 112 of the tube 45 so as to compress the elastic member 112 from its natural length along the central axis C of the elastic member 112. In contrast to this, when the pulling of the pulling member 44 is released by operating the lever 46 to move the swing base 52 from the swing position to the lowered position, one end 45a of the tube 45 moves in a direction to separate from the other end 45b along the central axis C of the elastic member 112. At this time, the compression force (compression) on the elastic member 112 of the tube 45 is gradually released to return the elastic member 112 to the natural length.

In this manner, as one end 45a of the tube 45 moves from the position shown in FIGS. 3A and 3B to the position shown in FIGS. 4A and 4B, one end 45a approaches the base 72 of the base member 62. The movement of the proximal end 45b of the tube 45 relative to the base 72 of the base member 62 is restricted. Accordingly, as the elastic member 112 of the tube 45 moves from the position shown in FIGS. 3A and 3B to the position shown in FIGS. 4A and 4B relative to the base member 62, the elastic member 112 is compressively deformed so as to shorten the total length. In this case, the elastic member 112 is formed such that the distal end side region 112a is more compressively deformable in a direction (lengthwise direction) along the central axis C of the elastic member 112 than the proximal end side region 112b. Accordingly, the distal end side region 112a of the elastic member 112 is greatly deformed, and the deformation amount of the proximal end side region 112b is smaller than that of the distal end side region 112a. When a compression force is exerted on the elastic member 112 so as to reduce the natural length, the proximal end side region 112b is deformed, for example, from the straight state shown in FIG. 3A to a state shown in FIG. 4A, in which the proximal end side region 112b is deformed into a wavy shape while the inner and outer diameters of the elastic member 112 are almost maintained. The proximal end side region 112b is deformed into a wavy shape without forming any creases. The distal end side region 112a is deformed from the straight state shown in FIG. 3A so as to form a plurality of creases without maintaining the inner and outer diameters of the elastic member 112 as shown in FIG. 4A. For this reason, the distal end side region 112a of the elastic member 112 of the tube 45 is easily deformed so as to be compressed along the central axis C of the pulling member 44 and the elastic member 112 of the tube 45 relative to the proximal end side region 112b and form continuous creases. That is, the elastic member 112 of the tube 45 is configured such that the distal end side region 112a as a region disposed between the distal end opening 82a of the first cylindrical surface 82 and the operating portion 42 is more deformable so as to be compressed along the central axis C of the tube 45 and form continuous creases than the proximal end side region 112b as a region disposed between the distal end 84a of the passage 84 and the distal end opening 82a of the channel hole 82. With this structure, creases are mainly formed on the distal end side region 112a of the elastic member 112, while the formation of creases on the proximal end side region 112b is suppressed.

When the operating portion 42 is moved from the lowered position shown in FIGS. 3A and 3B to the swing position (raised position) shown in FIGS. 4A and 4B, the movement amount of the pulling member 44 relative to the operating portion 42 (swing base 52 and coupling portion 54) is smaller than that of the pulling member 44 relative to the mouth ring 48a disposed on the base 72. Accordingly, the movement amount of the pulling member 44 relative to the elastic member 112 of the tube 45 gradually decreases as the pulling member 44 approaches the distal end of the elastic member 112 (one end 45a of the tube 45), and gradually increases as the pulling member 44 approaches the proximal end of the elastic member 112 (the other end 45b of the tube 45).

As described above, when the operating portion 42 is moved from the lowered position shown in FIGS. 3A and 3B to the swing position (raised position) shown in FIGS. 4A and 4B, a plurality of creases are formed on the distal end side region 112a of the elastic member 112 of the tube 45 as the distal end side region 112a is deformed so as to be compressed in the lengthwise direction. When a plurality of creases are formed on the distal end side region 112a of the elastic member 112, the outer circumferential surface of the pulling member 44 easily comes into contact with the inner circumferential surface of the distal end side region 112a of the elastic member 112. However, the movement amount of the pulling member 44 relative to the elastic member 112 of the tube 45 gradually decreases as the pulling member 44 approaches the distal end of the elastic member 112 (one end 45a of the tube 45). This minimizes the friction between the outer circumferential surface of the pulling member 44 and the inner circumferential surface of the distal end side region 112a of the elastic member 112.

On the other hand, this prevents the proximal end side region 112b of the elastic member 112 of the tube 45 from being deformed so as to be compressed in the lengthwise direction. Although the movement amount of the pulling member 44 relative to the operating portion 42 gradually increases as the pulling member 44 approaches the other end 45b from one end 45a, the clearance between the inner circumferential surface of the proximal end side region 112b and the pulling member 44 is maintained as needed. This minimizes the friction between the outer circumferential surface of the pulling member 44 and the inner circumferential surface of the proximal end side region 112b of the elastic member 112.

As described above, the proximal end side region 112b of the elastic member 112 of the tube 45 is arranged between the distal end 84a of the passage 84 and the distal end opening 82a of the first cylindrical surface (channel hole) 82 along the longitudinal axis L. That is, the proximal end side region 112b of the elastic member 112 of the tube 45 is arranged in the appropriate space 85 that allows the deformation of the proximal end side region 112b and is formed between the elastic member 112 and the inner circumferential surface 96b of the distal end cover 96. Accordingly, the deformation of the proximal end side region 112b of the elastic member 112 of the tube 45 is suppressed within the range of the space 85. Because the deformation of the proximal end side region 112b of the elastic member 112 of the tube 45 is suppressed, even if the proximal end side region 112b is deformed, the proximal end side region 112b is prevented from coming into contact with the distal end cover 96.

The distal end side region 112a of the elastic member 112 of the tube 45 is deformed by exerting a compression force on the elastic member 112 along the central axis C of the elastic member 112, and is also arranged between the distal end opening 82a of the first cylindrical surface 82 and the operating portion 42 along the longitudinal axis L. The distal end side region 112a of the elastic member 112 of the tube 45 is arranged in the appropriate space 77 that allows the deformation of the distal end side region 112a and is formed between the elastic member 112 and the inner circumferential surface 96b of the distal end cover 96. The extension surface 76d of the second convex portion 76 forming the space 77 is located below the first wall surface 92a on the distal end side of the second cylindrical surface 84 forming the space 85 on the drawing surface of each of FIGS. 3A to 4B. In addition, the defining surface 74b of the first convex portion 74 forming the space 77 is located on the right side of the drawing surface of each of FIGS. 3B and 4B relative to the second wall surface 92b on the distal end side of the second cylindrical surface 84 forming the space 85. Accordingly, the space 77 is formed larger than the space 85 in the up and down direction and the widthwise direction. As shown in FIG. 4A, a length La of a portion, of the distal end side region 112a of the elastic member 112 of the tube 45, on which creases are formed in a direction along the central axis (longitudinal axis) C of the elastic member 112 is smaller than a length Lb of the deformed portion of the proximal end side region 112b in a direction along the central axis (longitudinal axis) C. For this reason, when creases are formed on the distal end side region 112a of the elastic member 112 of the tube 45, even if the distal end side region 112a is expanded in the up and down direction and the widthwise direction, the distal end side region 112a of the elastic member 112 is restrained within the range of the space 77. Even if the distal end side region 112a of the elastic member 112 of the tube 45 is deformed so as to form a plurality of creases on the distal end side region 112a, the deformed distal end side region 112a is prevented from coming into contact with the first and second convex portions 74 and 76 of the base member 62 and the inner circumferential surface 96b of the distal end cover 96.

Even if the elastic member 112 of the tube 45 is compressed in a direction along the longitudinal axis and the proximal end side region 112b is formed into a wavy shape, the proximal end side region 112b of the elastic member 112 is restrained within the range of the space 85. Even if the proximal end side region 112b of the elastic member 112 of the tube 45 is deformed, the deformed proximal end side region 112b is prevented from coming into contact with the first and second wall surfaces 92a and 92b and the inner circumferential surface 96b of the distal end cover 96.

As described above, although the elastic member 112 is deformed in an axial direction of the elastic member 112 as the pulling member 44 moves along the axial direction of the elastic member 112, the elastic member 112 is prevented from wearing by rubbing against the pulling member 44.

Note that when the elastic member 112 is to be replaced due to wear, aging, and the like, the operating portion 42, the tube 45 fixed to the distal end 45a of the operating portion 42, and the proximal end 45b of the tube 45 are collectively detached from the base member 62 of the distal frame portion 22. As shown in FIG. 7, a new tube 45 is disposed outside the pulling member 44, and the distal end 45a of the tube 45 is properly attached to the operating portion 42. The operating portion 42 and the proximal end 45b of the tube 45 are collectively attached to the base member 62 of the distal frame portion 22.

As described above, the following can be said about the endoscope 10 according to this embodiment.

The distal end side region 112a of the elastic member 112 of the tube 45 covering the pulling member 44 is formed from a resin material that is softer and more compressively deformable along the central axis C of the elastic member 112 than the proximal end side region 112b. When the operating portion 42 is to be moved from the lowered position shown in FIGS. 3A and 3B to the swing position (raised position) shown in FIGS. 4A and 4B, the movement amount of the pulling member 44 relative to the elastic member 112 is set such that the movement amount of the pulling member 44 relative to the proximal end side region 112b is larger than that of the pulling member 44 relative to the distal end side region 112a. In this embodiment, although the movement amount of the pulling member 44 relative to the proximal end side region 112b of the elastic member 112 is large, the formation of creases on the proximal end side region 112b is suppressed. In addition, even if the movement amount of the pulling member 44 relative to the distal end side region 112a of the elastic member 112 is small and a plurality of creases are formed on the distal end side region 112a, the occurrence of friction between the pulling member 44 and the distal end side region 112a is suppressed. This minimizes friction between the pulling member 44 and the inner circumferential surface of the elastic member 112. Accordingly, when the pulling member 44 is moved to move the swing base 52 between the lowered position and the swing position, the load applied from the pulling member 44 onto the elastic member 112 can be reduced. Therefore, even when the pulling member 44 that actuates the operating portion 42 is repeatedly moved to the distal end side and the proximal end side along the axial direction, the movement of the pulling member 44 in the axial direction can be maintained in a proper state. In addition, because the wear of the tube 45 covering the outside of the pulling member 44 can be suppressed, the replacement time of the elastic member 112 can be delayed as compared in the past.

In this embodiment, in particular, an appropriate clearance is formed between the outer circumferential surface of the pulling member 44 and the inner circumferential surface of the elastic member 112. This makes it possible to maintain the proper movement of the pulling member 44 in the axial direction even if the pulling member 44 that actuates the operating portion 42 is repeatedly moved to the distal end side and the proximal end side along the axial direction.

This embodiment is configured to suppress deformation to form creases on the proximal end side region 112b of the elastic member 112 which is located close to the mouth ring 48a. This makes it possible to prevent the proximal end side region 112b of the elastic member 112 from being drawn toward the mouth ring 48a even when the swing base 52 is located at the swing position (raised position).

This embodiment is configured to mainly form creases on the distal end side region 112a of the elastic member 112 and reduce the formation of creases on the proximal end side region 112b. In this case, because the formation of the proximal end side region 112b is reduced, the space 85 (the portion of the elastic member 112 which accommodates the proximal end side region 112b) need not be formed as a large space. Accordingly, the distal end side region 112a of the elastic member 112 is formed so as to be easily compressively deformed in a direction along the central axis C relative to the proximal end side region 112b. This can prevent an increase in the size of the distal frame portion 22.

The elastic member 112 according to this embodiment, in particular, is configured such that creases are mainly formed on the distal end side region 112a located closer to one end 45a of the elastic member 112 than the opening 82a of the channel 36. A position where creases are formed is located in the large space 77 adjacent to the proximal end side of the swing base 52 in the state shown in FIG. 4A along the longitudinal axis L. This can reduce the influence of the deformation of the elastic member 112 of the tube 45 on the layout of the distal frame portion 22. It is, therefore, possible to prevent the elastic member 112 of the tube 45 from interfering with the inner circumferential surface of the distal end cover 96.

In this embodiment, the proximal end side fixing portion (proximal end 45b) of the tube 45 is formed closer to the proximal end side than the distal end opening 82a of the channel 36. This makes it possible to maximize the length of the elastic member 112 and reduce the deformation amount of the elastic member 112 per unit volume. It is, therefore, possible to suppress the deterioration of the elastic member 112 which is caused by deformation due to repeated swinging movement of the operating portion 42 using the swinging mechanism 38.

In this embodiment, the passage for the mouth ring 48a is inclined relative to the longitudinal axis L. A portion of the tube 45 which located between one end 45a and the other end 45b is bent as needed. This makes it possible to maximize the length of the elastic member 112 and reduce the deformation amount of the elastic member 112 per unit volume. It is, therefore, possible to suppress the deterioration of the elastic member 112 which is caused by deformation due to repeated swinging movement of the operating portion 42 using the swinging mechanism 38.

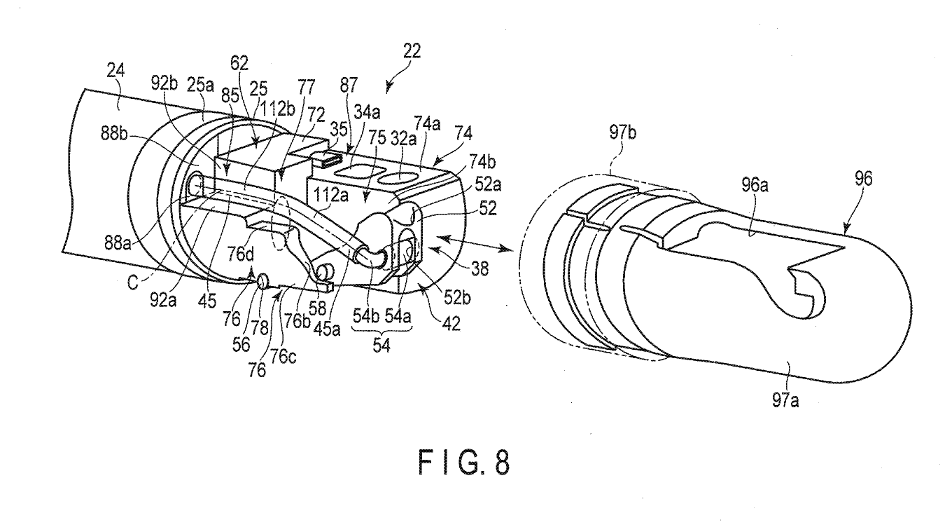

The above description has exemplified the case in which the distal frame portion 22 is formed with the distal end cover 96 being fixed to the base member 62. As shown in FIG. 8, the distal end cover 96 may be detachable with respect to the base member 62. Accordingly, the distal frame portion 22 need not necessarily include the distal end cover 96. When the distal end cover 96 is temporarily detached from the base member 62, a new distal end cover 96 having the same structure as that of the detached distal end cover 96 is preferably attached to the base member 62.

The above description has exemplified the case in which the other end 45b of the tube 45 is fitted in the mouth ring 48a fixed to the second cylindrical surface 84. Obviously, however, the other end 45b of the tube 45 may be directly fitted to the second cylindrical surface 84. That is, the other end 45b of the tube 45 may be directly bonded to the second cylindrical surface 84 without through the mouth ring 48a so as to ensure watertightness.

According to the above description, when the operating portion 42 is at the lowered position, the elastic member 112 has the natural length. However, the length of the elastic member 112 is not limited to the natural length. When the operating portion 42 is at the lowered position, the elastic member 112 may be extended or contracted as needed. When the lever 46 is at the first position, the elastic force of the pulling member 44, the tube 45, and the like act to suppress the unintentional movement of the lever 46 from the first position to the second position regardless of the state of the elastic member 112, thus preventing the operating portion 42 from unintentionally swinging.

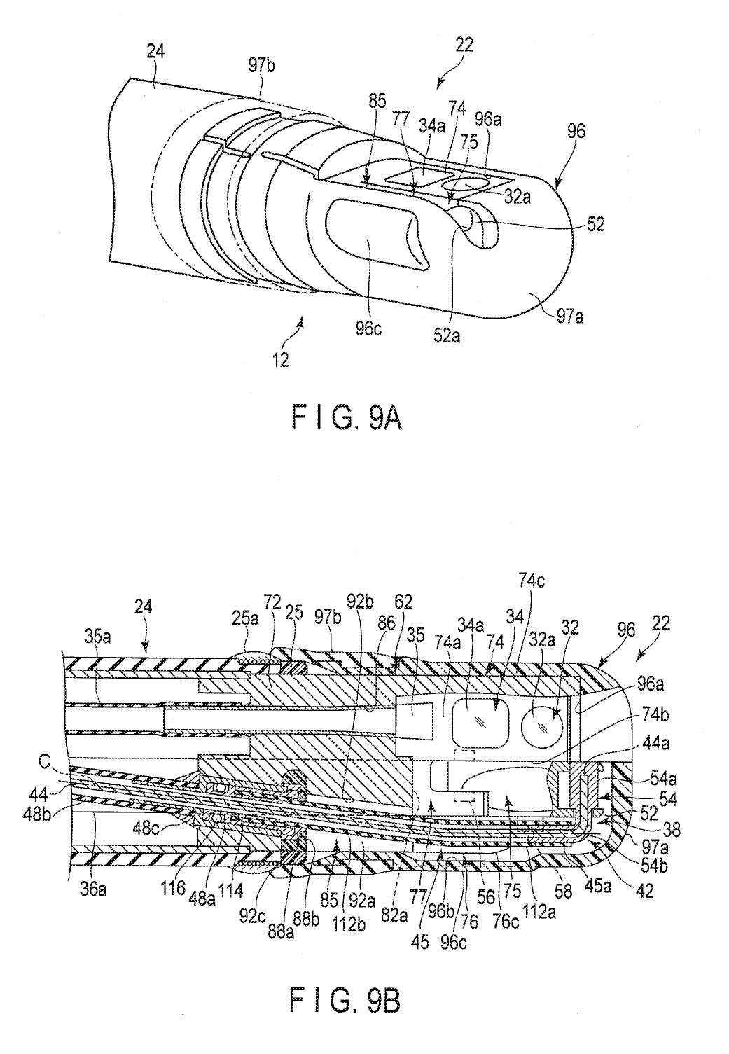

As shown in FIGS. 9A and 9B, the cover 96 includes a bulged portion 96c. The bulged portion 96c is formed at a position facing the distal end side region 112a of the elastic member 112. The bulged portion 96c shown in FIG. 9B makes the inner circumferential surface 96b of the cover 96 be arranged on the outside as compared with the case shown in FIG. 5. At this time, the thickness of the distal end cover 96 is preferably kept constant even in the presence of the bulged portion 96c.

At the swing position (raised position), the outer diameter of the elastic member 112 increases because of a plurality of creases formed on the distal end side region 112a of the elastic member 112. The bulged portion 96c makes the inner circumferential surface 96b of the cover 96 be arranged on the outside as in the case shown in FIG. 5. This suppresses the interference of the outer circumferential surface of the distal end side region 112a of the elastic member 112 of the tube 45 with the inner circumferential surface 96b of the cover 96. That is, only part of the distal end cover 96 may protrude outside in the radial direction relative to the longitudinal axis L.

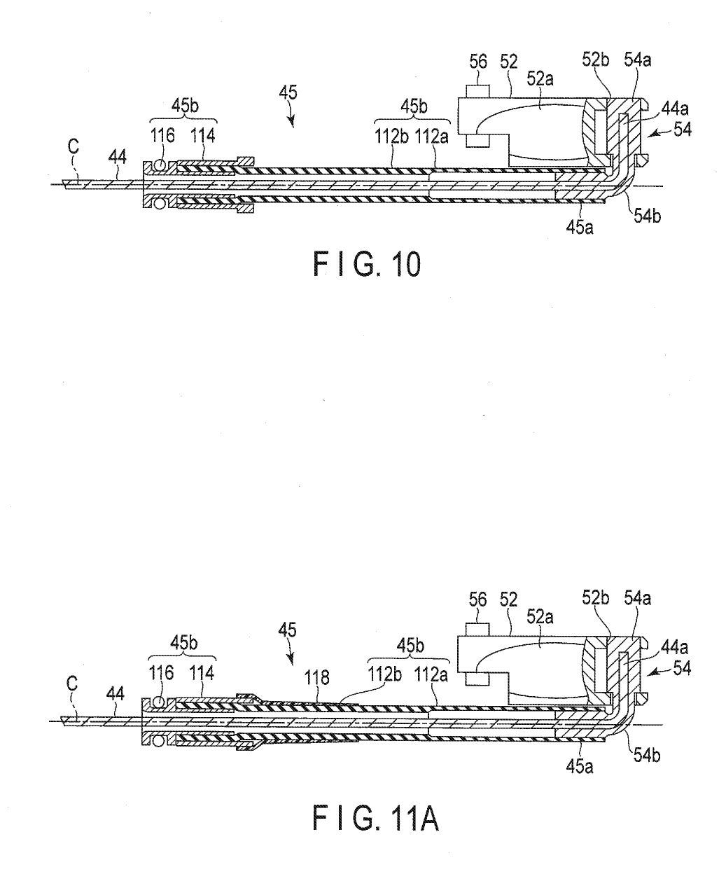

FIG. 10 shows the first modification of the tube 45 in which the pulling member 44 is disposed.

The elastic member 112, from the distal end to the proximal end, is formed from the same material. The distal end side region 112a of the elastic member 112 is formed thinner than the proximal end side region 112b. For the sake of simplicity, assume that in this case, the portion of the distal end side region 112a which is located between the distal end and the proximal end is formed to have the same thickness. In addition, for the sake of simplicity, assume that in this case, the portion of the distal end side region 112b which is located between the distal end and the proximal end is formed to have the same thickness. Note that the thickness of the distal end side region 112a is preferably reduced by about 10% to 30%, more preferably, about 20%, relative to the proximal end side region 112b. In this case, the thickness of the distal end side region 112a decreases at most to, for example, about 0.15 mm to 0.2 mm.

The compressive deformability of the elastic member 112 along the central axis C changes at the boundary between the proximal end of the distal end side region 112a and the distal end of the proximal end side region 112b. In this case as well, the distal end side region 112a is more compressively deformable in the lengthwise direction than the proximal end side region 112b. Accordingly, a side closer to one end 45a of the elastic member 112, connected to the operating portion 42 is more deformable than a side closer to the other end 45b of the elastic member 112, connected to the periphery of the passage 84.

The proximal end side region 112b is deformed from the straight state shown in FIG. 3A into a wavy shape while the inner and outer diameters of the elastic member 112 are maintained as shown in FIG. 4A. The distal end side region 112a is deformed from the straight state shown in FIG. 3A so as to form a plurality of creases on the distal end side region 112a without maintaining the inner and outer diameters of the elastic member 112 as shown in FIG. 4A. Therefore, creases are mainly formed on the distal end side region 112a of the elastic member 112, and the formation of creases on the proximal end side region 112b is suppressed.

Although the elastic member 112 of the tube 45 is formed as shown in FIG. 10 unlike in the first embodiment, the elastic member 112 of the tube 45 is deformed at a desired position relative to the base member 62 as shown in FIGS. 3A to 4B in the same manner as described in the first embodiment.

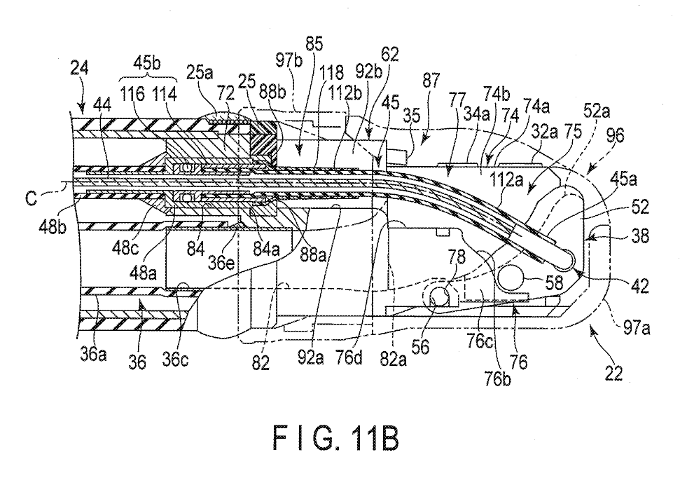

FIGS. 11A and 11B show the second modification of the tube 45 in which the pulling member 44 is disposed.

The tube 45 includes a reinforcing portion (reinforcing body) 118 in addition to the elastic member 112, the mouth ring 114, and the O-ring 116. The following will exemplify the reinforcing portion 118 having a cylindrical shape. However, the reinforcing portion 118 may have a helical shape or have a plurality of strip-shaped bodies extending along the longitudinal axis.

The following is a case in which the deformability of the material of the elastic member 112 itself is constant from the distal end to the proximal end. In this case, the deformability of the material of the elastic member 112 itself is preferably almost the same as that of the distal end side region 112a described in the first embodiment. As described in the first embodiment, the distal end side region 112a, of the elastic member 112, which is close to the one end 45a preferably has a property of being higher deformability than the proximal end side region 112b close to the other end 45b.

The reinforcing portion 118 is fixed to the mouth ring 114. The reinforcing portion 118 covers the outside of the proximal end side region 112b. The reinforcing portion 118 may be fixed to the outer circumferential surface of the proximal end side region 112b or may be simply in contact with or supported by the proximal end side region 112b. Fixing the reinforcing portion 118 to the outer circumferential surface of the proximal end side region 112b will make the reinforcing portion 118 easily exert the influence of its deformability on the proximal end side region 112b. The reinforcing portion 118 may be formed from a resin material having deformability similar to that of the distal end side region 112a of the elastic member 112 described in the first embodiment or a resin material less deformable than the distal end side region 112a. The reinforcing portion 118 acts cooperatively with the proximal end side region 112b of the elastic member 112 to reduce the deformability of the proximal end side region 112b of the elastic member 112. The reinforcing portion 118 covers the outside of the proximal end side region 112b to prevent the formation of ridges on the proximal end side region 112b and also prevent the formation of valleys originating from the formation of ridges on the proximal end side region 112b. Therefore, the elastic member 112 is formed such that the reinforcing portion 118 makes the distal end side region 112a close to one end 45a have higher compressive deformability along the central axis C of the elastic member 112 than the proximal end side region 112b close to the other end 45b.

In this manner, the reinforcing portion 118 prevents the proximal end side region 112b from being deformed into a wavy shape from the straight state shown in FIG. 3A while the inner and outer diameters of the elastic member 112 are maintained as shown in FIG. 4A. That is, a portion, of the proximal end side region 112b, which is covered by the reinforcing portion 118 maintains its straight state even when the operating portion 42 is located at the swing position (raised position). The distal end side region 112a is deformed from the straight state shown in FIG. 3A so as to form a plurality of creases on the distal end side region 112a without maintaining the inner and outer diameters of the elastic member 112 as shown in FIG. 4A.

When the reinforcing portion 118 is disposed on part of the proximal end side region 112b of the elastic member 112 of the tube 45, the proximal end side region 112b is less deformable than the distal end side region 112a as described in the first embodiment. In this case as well, when the operating portion 42 is located at the swing position (raised position), a plurality of creases are formed on the distal end side region 112a.

Note that the reinforcing portion 118, from the distal end to the proximal end, may be formed from the same material so as to have a constant thickness. The distal end side may be formed to be more deformable than the proximal end side.

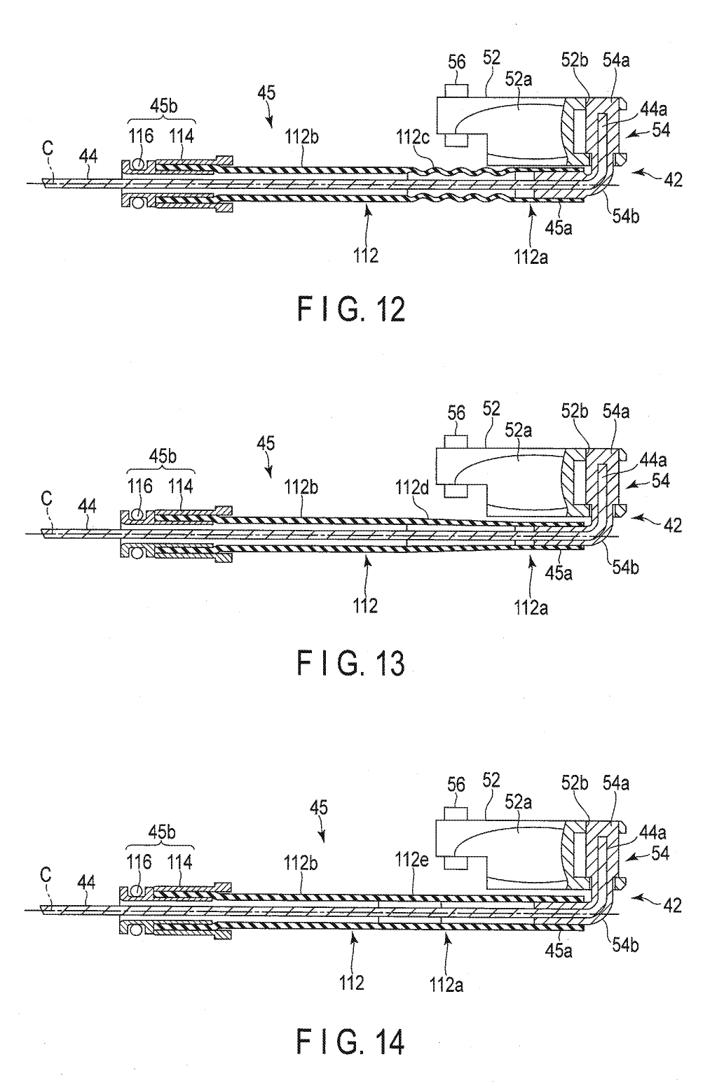

FIG. 12 shows the third modification of the tube 45 in which the pulling member 44 is disposed. FIG. 12 shows, in particular, a case in which the elastic member 112 of the tube 45 has a natural length.

The distal end side region 112a that is more compressively deformable along the central axis C of the elastic member 112 than the proximal end side region 112b includes, between the distal end and proximal end of the distal end side region 112a, a deformable portion 112c that is especially deformable. The deformable portion 112c is formed as a region which is most easily folded and on which a plurality of creases are easily formed when a compression force is exerted on the elastic member 112 along the central axis C. In this case, the deformable portion 112c is provided with a tendency to be formed into a wavy shape so as to allow easy visual recognition of the deformable region, even when the elastic member 112 of the tube 45 has a natural length. In this case, more specifically, although the deformable portion 112c of the distal end side region 112a of the elastic member 112 is the same as the other portion of the distal end side region 112a in terms of the composition and thickness of the material, but is provided with a tendency to be formed into a meander shape.

Although the elastic member 112 of the tube 45 is formed as shown in FIG. 12 unlike in the first embodiment, the elastic member 112 of the tube 45 is deformed in the same manner as described in the first embodiment. When a compression load is exerted on the elastic member 112 along the central axis C of the elastic member 112, the deformable portion 112c of the distal end side region 112a can be more reliably deformed to form a plurality of creases than the proximal end side region 112b.

FIG. 13 shows the fourth modification of the tube 45 in which the pulling member 44 is disposed.

The distal end side region 112a that is more compressively deformable along the central axis C of the elastic member 112 than the proximal end side region 112b includes, between the distal end and the proximal end of the 112a, a deformable portion 112d that is especially deformable. The deformable portion 112d is formed as a region which is most easily folded along the longitudinal axis and on which a plurality of creases are easily formed when a compression force is exerted on the elastic member 112 along the central axis C. In this case, the deformable portion 112d is formed so as to become thinner from the proximal end side to the distal end side.

Note that the thickness of the distal end side region of the deformable portion 112d of the distal end side region 112a is preferably reduced by about 10% to 30%, more preferably, about 20%, relative to the proximal end side region. In this case, the thickness of the deformable portion 112d of the distal end side region 112a decreases at most to, for example, about 0.15 mm to 0.2 mm.

Although the elastic member 112 of the tube 45 is formed as shown in FIG. 13 unlike in the first embodiment, the elastic member 112 of the tube 45 is deformed in the same manner as described in the first embodiment. When a compression load is exerted on the elastic member 112 along the central axis C of the elastic member 112, the deformable portion 112d of the distal end side region 112a can be more reliably deformed to form a plurality of creases than the proximal end side region 112b.

FIG. 14 shows the fifth modification of the tube 45 in which the pulling member 44 is disposed.

The distal end side region 112a that is more compressively deformable along the central axis C of the elastic member 112 than the proximal end side region 112b includes, near the boundary between the distal end side region 112a and the proximal end side region 112b, a deformable portion 112e that is especially deformable. The deformable portion 112e is formed as a region which is most easily folded along the longitudinal axis and on which a plurality of creases are easily formed when a compression force is exerted on the elastic member 112 along the longitudinal axis.

Although the elastic member 112 of the tube 45 is formed as shown in FIG. 14 unlike in the first embodiment, the elastic member 112 of the tube 45 is deformed in the same manner as described in the first embodiment. When a compression load is exerted on the elastic member 112 along the central axis C of the elastic member 112, the deformable portion 112e of the distal end side region 112a can be more reliably deformed to form a plurality of creases than the proximal end side region 112b.

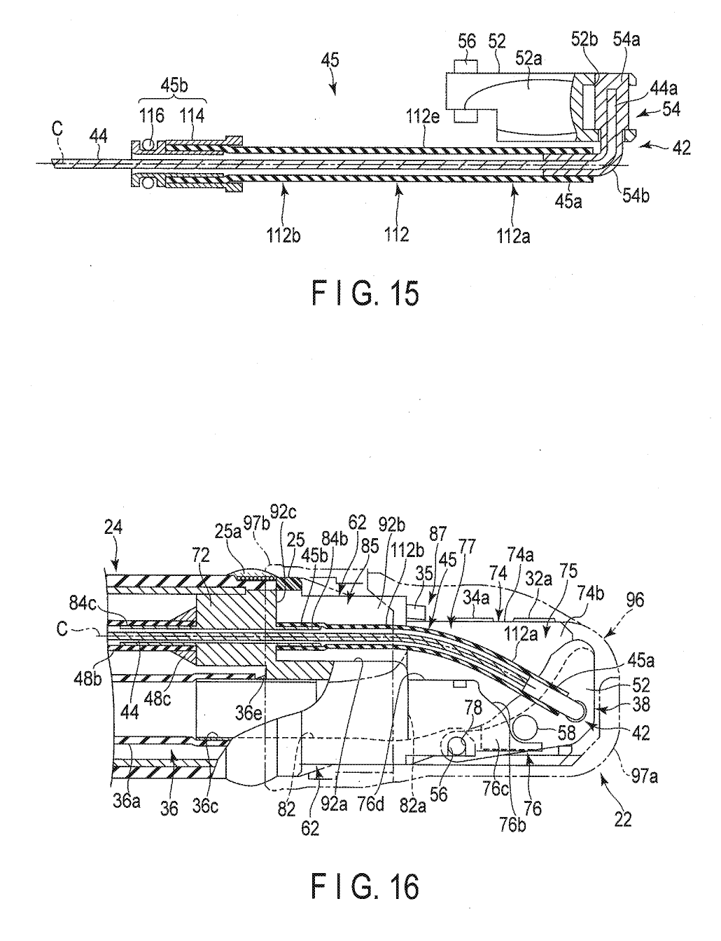

FIG. 15 shows the sixth modification of the tube 45 in which the pulling member 44 is disposed.

The flexibility of the elastic member 112 of the tube 45 gradually decreases from the proximal end to the distal end. The elastic member 112, in particular, is formed so as to gradually become compressively deformable from the proximal end to the distal end along the central axis C of the elastic member 112. That is, the elastic member 112 is formed such that a plurality of creases are formed more easily with a decrease in distance to the distal end side. That is, in this modification, the distal end side region 112a and the proximal end side region 112b of the elastic member 112 do not have a distinct boundary.

Although the elastic member 112 of the tube 45 is formed as shown in FIG. 15 unlike in the first embodiment, the elastic member 112 of the tube 45 is deformed in the same manner as described in the first embodiment. As described above, the elastic member 112 is formed so as to gradually become compressively deformable from the proximal end to the distal end along the central axis C of the elastic member 112 when a compression load is exerted on the elastic member 112 along the central axis C of the elastic member 112. This makes it possible to deform the elastic member 112 so as to form a plurality of creases more on the distal end side region than the proximal end side region.

FIG. 15 shows a case in which the elastic member 112 has a constant thickness but changes in flexibility from the proximal end to the distal end. In addition, although the composition of the material for the elastic member 112 remains the same from the proximal end to the distal end, it is obvious that the elastic member 112 may be tapered by gradually reducing the thickness of the elastic member 112 from the proximal end to the distal end.

The second embodiment will be described with reference to FIG. 16. This embodiment is a modification of the first embodiment including the above modifications. The same reference numerals denote the same members or members having the same functions as those described in the first embodiment when possible, and a detailed description of the members will be omitted.

As shown in FIG. 16, on a second cylindrical surface 84 forming the second through hole of a base 72, a mouth ring 84b protruding toward a space 85 is integrally molded with the base 72 of a base member 62. Referring to FIG. 16, a mouth ring 84c protruding toward the proximal end side is integrally molded with the base 72 of the base member 62. The mouth ring 84b and the mouth ring 84c of the second cylindrical surface 84 are formed as a passage for making the distal end side communicate with the proximal end side.

Note that the mouth ring 84b may be integrally molded with the distal end of the mouth ring 48a described in the first embodiment. That is, the mouth ring 84b may be fixed to the base 72 of the base member 62.

The proximal end portion of an elastic member 112 of a tube 45 in which a pulling member 44 is disposed is fixed to the mouth ring 84b. Although not shown, the inner circumferential surface of a proximal end 45b of the elastic member 112 is entirely in tight contact with the outer circumferential surface of the mouth ring 84b of a proximal end 45b of the elastic member 112 with, for example, an adhesive agent. This prevents a liquid or gas from infiltrating the inside of the elastic member 112 from the proximal end (the other end) 45b of the elastic member 112 of the tube 45. With this structure, the other end 45b of the tube 45 is watertightly connected to the base member 62 so as to have a structure for preventing a liquid from infiltrating the proximal end side through the passage (the mouth ring 84b, the second cylindrical surface 84, and the mouth ring 84c) and the base 72 of the base member 62. In this manner, the proximal end 45b of the tube 45 may be fixed at a position closer to the distal end side than a third wall surface 92c of the base 72 along the longitudinal axis L.

In the case shown in FIG. 16, an adhesive agent 88a and a retaining plate 88b are not required on the condition that the other end 45b of the tube 45 is kept fixed to the mouth ring 84b.

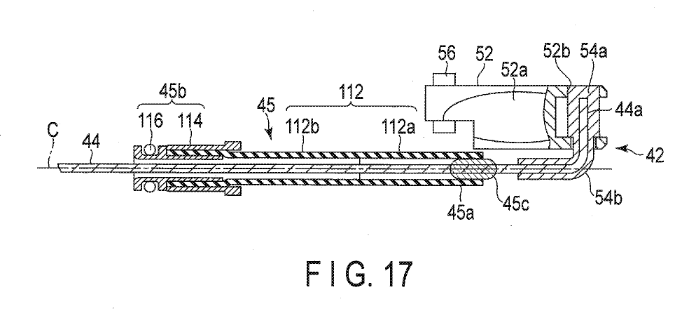

The third embodiment will be described with reference to FIG. 17. This embodiment is a modification to the first and second embodiments including the above modifications. The same reference numerals denote the same members or members having the same functions as those described in the first and embodiments when possible, and a detailed description of the members will be omitted.

The following is a case in which a distal end 45a of an elastic member 112 of a tube 45 in which a pulling member 44 is disposed is directly fixed to the pulling member 44 instead of a tubular body 54b of a coupling portion 54. The distal end 45a of the elastic member 112 of the tube 45 is preferably fixed to the pulling member 44 by an adhesive agent 45c at a position near the tubular body 54b of the coupling portion 54. The inner circumferential surface of the distal end 45a of the elastic member 112 is entirely in tight contact with the outer circumferential surface of the pulling member 44 with the adhesive agent 45c. This prevents a liquid or gas from infiltrating the inside of the elastic member 112 from the distal end (one end) 45a of the elastic member 112 of the tube 45. With this structure, the distal end 45a of the elastic member 112 of the tube 45 is watertightly connected to the pulling member 44. The distal end 45a of the elastic member 112 of the tube 45 is fixed to the pulling member 44 at a position near the tubular body 54b of the coupling portion 54 in order to minimize the exposure of the pulling member 44. Note that a proximal end 45b of the tube 45 is watertightly connected to a base member 62. In the same manner as described above, the tube 45 prevents a liquid from infiltrating the proximal end side from the distal end side of a passage (for example, a mouth ring 48a) through the passage (for example, the mouth ring 48a).

As described above, a portion, of the elastic member 112 of the tube 45 according to this embodiment, which extends between the distal end (one end) 45a and the proximal end (the other end) 45b is deformable, and the side of the distal end side region 112a closer to the distal end 45a is more compressively deformable along a central axis C of the tube 45 than the side of the proximal end side region 112b closer to the proximal end 45b.

A distal end side region 112a of the elastic member 112 of the tube 45 is preferably disposed between a distal end opening 82a of a first cylindrical surface 82 and an operating portion 42 while a swing base 52 is moved to the swing position (the maximum swing position or maximum raised position) shown in FIG. 4A. That is, a position where the distal end side region 112a of the elastic member 112 is compressively deformed along the axial direction of the elastic member 112 to form a plurality of creases is preferably located between the distal end opening 82a of the first cylindrical surface 82 and the operating portion 42. As described above, when the pulling member 44 is pulled to the proximal end side and the swing base 52 is arranged at the swing position, the distal end 45a of the tube 45 is located closer to the distal end side of the base member 62 than the distal end opening 82a of the first cylindrical surface 82 forming the first through hole (channel hole). Accordingly, the distal end 45a of the elastic member 112 of the tube 45 is preferably fixed at a position as close to a distal end portion 44a of the pulling member 44 as possible. For example, the distal end 45a of the elastic member 112 of the tube 45 is preferably located at a position in contact with or close to the coupling portion 54. When, in particular, the distal end 45a of the elastic member 112 of the tube 45 comes into contact with the proximal end of the tubular body 54b of the coupling portion 54, the length of the elastic member 112 can be maximized, and the elastic member 112 can be made compressively deformable along the central axis C.

As described above, the elastic member 112 of the tube 45 need not be directly connected to the operating portion 42.

The first to third embodiments including the above modifications each have exemplified the observation optical system 34 as a side-viewing type. A known operating portion 42 can be used for an endoscope 10 including a direct-viewing observation optical system 34, and a known operating portion 42 can be used for an endoscope 10 including an oblique-viewing observation optical system 34. The movement amount of the pulling member 44 that actuates the operating portion 42 of the endoscope 10 including the direct-viewing or oblique-viewing observation optical system 34 is small near operating portion 42 and gradually increases with a decrease in distance to a second cylindrical surface 84 of the base member 62. This is the same as in the above embodiments. The side closer to one end 45a of the elastic member 112 of the tube 45 is made more compressively deformable along the central axis C of the tube 45 than the side closer to the other end 45b of the elastic member 112. This is the same as in the above embodiments. Accordingly, the tube 45 can be used in the same manner for not only the side-viewing endoscope 10 but also the direct-viewing and oblique-viewing endoscopes 10.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope comprising:
a base that is provided on a distal end portion of an insertion portion, and that includes a passage configured to communicate a distal end side with a proximal end side;
an operating portion that is provided on a distal end side of the passage and is configured to be actuated with respect to the base;
an elongated member that includes a distal end side connected to the operating portion, and a proximal end side extending to a proximal end side of the base through the passage, and is configured to actuate the operating portion by moving in an axial direction of the elongated member; and
a tube into which the elongated member is inserted,
the tube including one end watertightly connected to the operating portion or the elongated member, and the other end watertightly connected to the base, the tube being configured to prevent a liquid from infiltrating the proximal end side of the passage from the distal end side of the passage through the passage,
the tube being elastically deformable between the one end and the other end of the tube, and
the tube being formed from a resin material, a side closer to the one end of the tube being more compressively deformable along a longitudinal axis of the tube than a side closer to the other end of the tube.

2. The endoscope of claim 1, wherein:
the base includes a channel hole through which a treatment tool is configured to be inserted, and
the distal end of the passage of the base is located closer to the proximal end of the base with respect to a distal end opening of the channel hole.

3. The endoscope of claim 2, wherein:
the base includes a support portion that is configured to support the operating portion so as to allow the operating portion to operate, and
the operating portion includes a swing base that is configured to operate around the support portion with a pulling force from the elongated member and is configured to change a direction of a distal end of the treatment tool protruding from the distal end opening of the channel hole.

4. The endoscope of claim 1, wherein the other end of the tube is fitted in the passage.

5. The endoscope of claim 1, wherein:
the passage includes a mouth ring extending to a distal end side of the base, and
the other end of the tube is fixed to the mouth ring.

6. An endoscope comprising:
a base that is provided on a distal end portion of an insertion portion, and that includes a passage configured to communicate a distal end side with a proximal end side;
an operating portion that is provided on a distal end side of the passage and is configured to be actuated with respect to the base;
an elongated member that includes a distal end side connected to the operating portion, and a proximal end side extending to a proximal end side of the base through the passage, and is configured to actuate the operating portion by moving in an axial direction of the elongated member; and
a tube into which the elongated member is inserted,
the tube including one end watertightly connected to the operating portion or the elongated member, and the other end watertightly connected to the base, the tube preventing a liquid from infiltrating the proximal end side of the passage from the distal end side of the passage through the passage,
the tube being elastically deformable between the one end and the other end, and
a side of closer to the one end of the tube having a smaller thickness than a side closer to the other end of the tube, and the side closer to the one end of the tube being more compressively deformable along a longitudinal axis of the tube than the side closer to the other end of the tube.

7. The endoscope of claim 6, wherein:
the base includes a channel hole through which a treatment tool is configured to be inserted, and
the distal end of the passage of the base is located closer to the proximal end of the base with respect to a distal end opening of the channel hole.

8. The endoscope of claim 7, wherein:
the base includes a support portion that is configured to support the operating portion so as to allow the operating portion to operate, and
the operating portion includes a swing base that is configured to operate around the support portion with a pulling force from the elongated member and is configured to change a direction of a distal end of the treatment tool protruding from the distal end opening of the channel hole.

9. The endoscope of claim 6, wherein the other end of the tube is fitted in the passage.

10. The endoscope of claim 6, wherein:
the passage includes a mouth ring extending to a distal end side of the base, and
the other end of the tube is fixed to the mouth ring.

11. An endoscope comprising:
a base that is provided on a distal end portion of an insertion portion, and that includes a passage configured to communicate a distal end side with a proximal end side;
an operating portion that is provided on a distal end side of the passage and is configured to be actuated with respect to the base;
an elongated member that includes a distal end side connected to the operating portion, and a proximal end side extending to a proximal end side of the base through the passage, and is configured to actuate the operating portion by moving in an axial direction of the elongated member; and
a tube into which the elongated member is inserted,
the tube including one end watertightly connected to the operating portion or the elongated member, and the other end watertightly connected to the base, the tube being configured to prevent a liquid from infiltrating the proximal end side of the passage from the distal end side of the passage through the passage,
the tube being elastically deformable between the one end and the other end, and
the tube including, on an outside of the tube, a reinforcing body that is configured to change deformability of a region between the one end of the tube and the other end, a side closer to the one end of the tube being more compressively deformable along a longitudinal axis of the tube than a side closer to the other end of the tube.

12. The endoscope of claim 11, wherein:
the base includes a channel hole through which a treatment tool is configured to be inserted, and
the distal end of the passage of the base is located closer to the proximal end of the base with respect to a distal end opening of the channel hole.

13. The endoscope of claim 12, wherein:
the base includes a support portion that is configured to support the operating portion so as to allow the operating portion to operate, and
the operating portion includes a swing base that is configured to operate around the support portion with a pulling force from the elongated member and is configured to change a direction of a distal end of the treatment tool protruding from the distal end opening of the channel hole.

14. The endoscope of claim 11, wherein the other end of the tube is fitted in the passage.

15. The endoscope of claim 11, wherein:
the passage includes a mouth ring extending to a distal end side of the base, and
the other end of the tube is fixed to the mouth ring.
